(19) United States
(12) Patent Application Publication
(10) Pub. No.: US 2022/0354682 A1
(43) Pub. Date: Nov. 10, 2022

(54) IMPROVED ORTHOPEDIC VENTING TOTAL CONTACT CAST TO REDUCE EDEMA AND SUFFOCATION

(71) Applicant: ALESSANDRO BARBERIO, AURORA (CA)

(72) Inventor: ALESSANDRO BARBERIO, AURORA (CA)

(21) Appl. No.: 17/621,836

(22) PCT Filed: Jun. 24, 2020
(86) PCT No.: PCT/CA2020/000074

§ 371 Date: Dec. 22, 2021

Related U.S. Application Data
(60) Provisional application No. 62/921,564, filed on Jun. 25, 2019.

(51) Int. Cl. A61F 5/01; A61F 13/04

(52) U.S. Cl. CPC A61F 5/0111; A61F 13/04

(57) ABSTRACT

The invention provides an improved method for installing a total cast lower leg to a patient to reduce edema and suffocation and to pro-vide a ventilation to the wounded limb. The method includes covering the swollen patient's limb with a number of different layers resulting in creating an edema shock absorber device, which facilitates the removal of an excessive amount of heat, bad smell, bacteria, germs and gazes outside of the cast.

### FIELD OF THE INVENTION

Charcot's arthropathy and Neuropathic ulcer with edema: Charcot's arthropathy (inflammation of joints) and neuropathic ulcers (damage to the peripheral nerves) are diseases that require the use of an off-loading brace as a mean to off-load the wounded limb and be able to ambulate at the same time. Total contact casts assembly or embodiment is the traditional key to cure these above indicated diseases.

Edema takes place when the leg is inside a total contact cast. Therefore this (edema) is a serious scenario because it is lengthy and complicated. Good results are not always achieved because arthropathy and neuropathy are not easy to cure in a short period of time. No guaranties for final cures depending on the progress and health of each patient.

Other Injuries with Edema

In the first 48 hours to 72 hours after an injury, an injured joint may swell, causing the limb to feel uncomfortably tight or snug. This is called “edema” and it could happen also to animals. The casted limb may become swollen, painful, hot, and inflamed. Almost all actual immobilizing cast for limbs they tend to become too tight around the casted or wounded limb. The limb becomes painful, hot, itchy and in certain cases smelly, leading to maceration and itchiness. The rehabilitation period of time is abnormally extended and the cost of recasting the limb including prescribed medication puts an additional load or burden of unwanted financial side effects towards our medical system. The patient becomes less energetic and productive as well. There are certain things to do in order to alleviate the consequences of edema as if elevation of the injured limb. Positioning the injured limb above the heart, using pillows or other support. Elevation encourages to drain out of the injured limb, easing swelling. Also, by icing the cast while the cast is elevated is second remedy. Sometimes, these solutions work but it takes time until the edema pressure decreases fully. Every patient is fully different and recovery time is short or long or not at all.

No matter how short or long is the timing for decreasing the edema effects but during that time some patients are very disturbed and can hardly adapt to the suffering of edema. Especially if the patient is frail And poor in health, any flaring edema or short lived one will cause discomfort and unbearable suffering accompanied by psychological discomfort.

In the past I already mentioned all these above indicated problems excluding edema, and I designed few orthopedic patents related to having “ventilated or aerated” surgical casts.

This was included inside my PCT Patent Application Number: PCT/CA2018/051668 by title “Improved tubular venting devices for surgical casts and other orthopedic devices”.

Edema occurs to patients having immobilized limbs by surgical fiber glass casts and by plaster of Paris casts. So far patients that have ulcers underneath the foot they are receiving TCC (total contact cast system of immobilization. This system allows the wounded area to bear/endure less body weight. Therefore, the TCC will reduce the pressure exerted on the foot by reducing the amount of pressure exerted on the foot by reducing the amount of pressure on the affected area and it redistributes the localized high plantar pressures to a larger surface area.

The whole process is clearly explained inside the U.S. Pat. No. 6,974,431 132. Charcot Neuroarthropathy renders unfortunately the foot insensate. Now if a patient has all these above indicated problems and the insensation feeling this is or will be already a serious problem. But actually, we are forgetting the IDEMA PROBLEM too! It is a silenced one. So far no patents were issued in order to alleviate orthopedically or prosthetically this above indicated problem. The only rapid solution is medication and recast the patient's limb. With the new proposed teaching we will save the future expense to install a new cast later on because with the new design we will help the wounded skin to experience less painful pressure around it. This new TCC will minimize the amount of sweat, odors, itchiness, bacteria.

This suggested device will reduce the expenses for the patient and for the insurance system because the rehabilitation phase will be shorter and more comfortable. It is a known fact that not all orthopedic devices are correcting completely all problems without leaving a negative impact to the patient who is using those devices. Edema is a common problem caused to immobilized limbs by all kinds of orthopedic casts.

It is suggested a new solution with an improved design helping the patient feel less compression by edema. This new orthopedic apparatus will be implemented to existing and future immobilizing casts.

Existing total contact casts do not focus on reducing edema.

Edema is a radiant force as a vector that pushes permanently the skin of the injured limb against the internal hard walls of the fiberglass cast.

This situation causes a painful discomfort and psychological distress. The edema force must be deviated. This must be done by using a new Edema membrane called the new orthopedic “shock absorber”.

### REFERENCES CITED

Improved tubular venting device for surgical casts and other orthopedic devices. PCT Application No. PCT/CA2018/051668
Total contact cast U.S. Pat. No. 9,192,504B2 2013 Apr. 17
Total contact cast system US20180177642A1 2016 Dec. 27
Apparatus and method for applying a total contact cast 6974431 B2 2003 Apr. 24

### BACKGROUND OF THE INVENTION

The invention is directed to a surgical orthopedic cast and more specifically to an internal set of compressible layers designed to maximize edema release, reduce bad smell, excessive presence of sweat and bacteria.

The invention is directed to total contact casts for neuropathic foot to be swollen by edema.

The invention is directed to improvement of tubular casts for upper limbs as the PCT Application No. PCT/CA2018/051668 by title “Improved tubular venting device for surgical casts and other orthopedic devices” to be swollen by edema.

The invention is directed to improvements of fiberglass casts to be wrapped around lower limbs to be swollen by edema.

The invention is directed to improvements of fiberglass casts to be wrapped around upper limbs to be swollen by edema.

The invention is directed to improvements of fiberglass casts to cure Charcot Neuroarthropathy to be swollen by edema.

Edema affects lower and upper immobilized limbs by fiberglass casts and by plaster of Paris casts at certain times and periods.

During edema patients suffer and casts are removed in order to replace them by larger ones costing the system more money.

Edema causes a painful and longer rehabilitation for the patients.

Edema could tempt the patient to introduce sharp objects inside the fiberglass cast and cause further injuries.

So far, no new immobilizing casts was introduced in order to decrease the pressure of edema when a limb is stuck inside a fiberglass cast for long time. The risk of edema is of course always a risk with a cast due to the possibility of impaired circulation. Skin damage from edema's persistent pressure may be worse from damage from friction and sheer inside immobilizing casts.

Neuropathic ulcers foot with edema could strike the following areas as:

a. Over toe joints.
b. Under metatarsal head.
c. Inner side of metatarsal head.
d. Under heel.
e. Over malleoli.

This invention teaches the construction of a shock absorber device used against painful edema without use of drugs when injected to a wounded limb.

This invention applies to a swollen limb by edema a traditional cotton layer to absorb sweat, exudate, bacteria and bad smells. Considered as a first layer.

This invention applies in transferring the absorbed sweat, exudate, bacteria, bad smells and excessive heat towards a tubular cotton stockinette. Considered as second layer.

This invention applies in transferring the absorbed sweat, exudate, bacteria, bad smells and excessive heat from a tubular cotton stockinette towards a multi-perforated of thermoformed polymer as an EVA or Plastazote material. Considered as third layer.

This invention applies in using an EVA or Plastazote material made of three to four layers with each layer to carry a distinct hardness of Shore A.

This invention applies in using an EVA or Plastazote material made of four layers.

This invention applies in using the first EVA or Plastazote layer of hardness of Shore A as 10 to 20.

This invention applies in using the second EVA or Plastazote layer of hardness of Shore A as 25 to 30.

This invention applies in using the third EVA or Plastazote layer of hardness of Shore A as 35 to 36.

This invention applies in using the fourth EVA or Plastazote layer of hardness of Shore A as 37 to 38.

This invention applies in transferring the compressive force of edema towards the EVA or Plastazote layers.

This invention applies in using the layers of EVA or Plastazote to be fully perforated from first layer to last layer.

This invention applies in allowing the edema pressure to be transmitted first to the first layer of EVA or Plastazote where pressure of edema is ready to compress a first layer of EVA or Plastazote of maximum hardness of Shore A as 10 to 20.

This invention applies in allowing the edema pressure to be transmitted secondly to second layer of EVA or Plastazote where pressure of edema is ready to compress a second layer of EVA or Plastazote of maximum hardness of Shore A as 25 to 30.

This invention applies in allowing the edema pressure to be transmitted thirdly to third layer of EVA or Plastazote of maximum hardness of Shore A as 35 to 36.

This invention applies in allowing the edema pressure to be transmitted fourthly to fourth layer of EVA or Plastazote of maximum hardness of Shore A 37 to 38.

This invention applies in allowing the gases, the bad smell, the excessive heat, the condensed sweat to travel from first layer of EVA or Plastazote to last or fourth layer of EVA or Plastazote by means of perforations or pores.

This invention applies in transmitting all the excessive heat, bacteria, gazes, distilled oils and liquids, bad smells from above indicated quadruple layer of EVA or Plastazote via perforations towards a bumpy, perforated layer of EVA or Plastazote. Considered as a fourth layer.

This invention applies in allowing the bad smells, bacteria and excessive heat to travel between the protrusion and escape from inside the cast to the outer space.

This invention applies in the creation of a venting system inside an immobilizing cast.

This invention applies in having a bumpy, domed layer of hardness of Shore A 37-38 ready to absorb a maximum compressive edema force. Considered as fourth layer.

This invention applies in having a layer with domes with each dome with a protrusion.

This invention applies in having a polymer of Shore A 37-38 with a porous and multiperforated surface.

This invention applies in having a polymer of Shore A 37-38 made of domes of a base half by half inch.

This invention applies in having a polymer of Shore A 37-38 with domes separated by straight channeled grooves.

This invention applies in having a polymer of Shore A 37-38 with the physical characteristic to withstand the edema pressure of the swollen limb without collapsing or obstructing in within the perforations among its surface. Thus, allowing a free traffic in within its bumps of the bacteria, gazes, heat, and introduction of fresh air.

This invention applies in having a polymer of Shore A 37-38 with a dome and with a protrusion on the top of the dome all made by a thermoforming procedure.

This invention applies in having a polymer of Shore A 37-38 with a dome and its protrusions on top of each dome all made with thin walls of 1.5 to 2.0 mm.

This invention applies in having a polymer of Shore A 37-38 with a dome and its protrusions on top of each dome all made with distanced holes of 0.5 mm. diameter.

This invention applies in having a polymer of Shore A 37-38 with a dome and its protrusions on top of each dome all made with distanced holes of 0.5 mm. of diameter and its holes distanced by each other by 6 to 8 mm.

This invention applies in having a polymer of Shore A 37-38 with a dome and its protrusions on top of each dome with empty space inside each dome and inside each protrusion to allow the presence of ventilating air.

This invention applies in having a polymer of Shore A 37-38 with an empty protrusion and dome to allow to have a lighter membrane and finished product.

This invention applies in having a polymer of Shore A 37-38 with domes separated by each other with grooved channels in order to make the device more flexible during its application to the patient's limb.

This invention applies in having a polymer of Shore A 37-38 with domes and protrusions on top of the dome without collapsing from the edema force.

This invention applies in having a flat, perforated polymer on top of the protrusions of the domes of Shore A 37-38. Considered as fifth layer.

This invention applies in having a flat, perforated polymer on top of the protrusions of the domes of Shore A 37-38 facing the internal walls of the fiber glass cast or the cotton that is facing optionally the internal walls of the fiber glass cast.

This invention applies in having a flat, perforated polymer on top of the protrusions of the domes of Shore A 37-38 with holes of diameter smaller than the diameter of the protrusions.

This invention applies in having an external layer of fiber glass of plaster of Paris casts considered as sixth layer.

This invention applies in having the force of the edema compression of the swollen skin of the upper or lower limb to be absorbed by the third layer of the EVA polymer or Plastazote thus, without causing the need to assemble around the swollen limb a larger immobilizing cast.

### DETAILED DESCRIPTION OF THE PREFERRED

### Embodiments

FIG. 1. Casted skin affected by edema pressure.

FIG. 1. Is a cross section view a limb's skin affected by edema, being immobilized by a surgical fiberglass cast and cushioned by protective polymeric layers.

### First Layer (I)

The skin of an immobilized limb is shown as (1) being affected by edema force as indicated by arrows (2).

The direction of the edema pressure (2) against the internal walls of immobilizing cast (15) is shown by arrows (2).

The wound is in (3) covered by a medicated wound dressing as in (3b.).

The bacteria and different sources of vapours as (9) exit from the surface of the casted skin (1) towards and underneath the open sides of the cast at (16).

### Second Layer (II):

The limb (1) is optionally covered by a sterilized and porous cotton layer as in (5).

### Third Layer (III):

The whole limb (1) is covered by a surgical hydrophilic sterilized expandable tubular stockinette (6).

### Fourth Layer (IV):

A mono or dual or triple or quadruple or laminated layered polymer of EVA or Plastazote shown as (7) is installed on top of the stockinette (6).

The quadruple layer (7) has engraved channels on top of its first upper layer (D).

The quadruple layer (7) has engraved channels on its layer (D) representing the shape of squares or rectangles or lozenges or circles.

This vast choice of different shapes and sizes will make the membrane (7) more easily adaptable or flexible in order to be installed around an upper or lower wounded limb.

The engraved channels are shown as 8.

The quadruple or triple laminated layered polymer of EVA or Plastazote (7) has four layers laminated to each other being all perforated directly from the first to the last layer with perforations as in (8a.).

The laminated layer (7) is made of the following layers each having a distinct Shore Hardness A as it follows:

First layer as in (A) has a hardness of Shore A from 20 to 25. Layer (A) is in direct contact with stockinette (7).

Second layer as in (B) has a hardness of Shore A from 25 to 30.

Third layer as in (C) has a hardness of Shore A from 30 to 35.

Fourth layer as in (D) has a hardness of Shore A from 35 to 40

Membrane (7) has the unique function to absorb gradually the force of compression generated from the edema of the wounded limb.

This absorption of edema force is stored inside the layer (A) then if this force is too big to be handled by layer (A) it is consequently passed to other layers belonging to quadruple membrane (7).

This absorption of edema force is stored inside the layer (A) then as an example, it is stored inside layer (B) and as its magnitude of compression increases it will be transmitted to layer (C) and finally layer (D).

Membrane (7) will absorb as an absorbed energy generated by the edema force (2) of compression of the wounded limb.

This above indicated stored compressed energy (2) by being stored inside membrane (7), automatically it will spare the wounded limb from being asphyxiated or compressed harshly against the hard walls of the immobilizing fiberglass cast.

This membrane (7) is a protector against the suffocating force of edema, and it spares the need to replace the immobilizing cast by a second and larger one.

It will save time, more medical expenses and avoid of course a risk due to the possibility of impaired circulation.

It will avoid of course a risk due to the possibility of impaired circulation.

Persistent pressure is worse from friction and sheer inside a cast.

Perforated layer (7) is considered to be as a gentle “shock absorber” or a tool for edema relief making the lives of the patients more comfortable for many weeks of casting immobilization.

In other words the membrane (7) will be compressed by the edema force and by this method the wounded skin (1) will not be compressed internally against the internal walls of the cast (15).

Perforated layer (7) allows the excessive heat, the bacteria indicated as micro particles as 9, the bad smells, the gazes, the condensed vapours, all of them to escape the wounded skin 1 via its holes 8.a.

Layer (7) is porous thus allowing more of all the above stated unwanted elements to escape the wounded area.

Layer (7) is perforated by holes 8.a and it is also invisibly porous.

Layer (7) can be made from only one layer up to four layers.

The hardness of each layer can be chosen in accordance of the patient's needs.

Layer (7) can be made of different materials combined together in accordance of the mechanical and chemical requirements.

EVEA membrane, or and silicone membrane, or and gel membrane, or and plastazote™ membrane might be applied all together.

Layer (7) must include at least one compressible layer.

Existence of compressibility characteristic of at least one layer is a must for edema reduction.

### Fifth Layer (V):

Made of EVA™ or medical silicone or Plastazote™ as a thermoformed polymer as layer (10a.)

Layer (10a.) with protrusions as in (11).

The protrusions (11) are made of solid material.

Protrusions (11) are solid made without an empty cavity.

This is in order to be able to withstand any compressive pressure coming from the swollen skin 1 and from the pressure of the external applied immobilizing cast indicated as 15.

The layer (10.a) has perforations as (10).

Perforations (10) are scattered over fifth layer (10a) in order to allow the passage of evaporated gases as sweat, or oils, or vapours, or pus, or blood to travel between the empty spaces that exist between the protrusions (11) and to exit the immobilizing fiberglass cast (15).

Protruded fifth layer (10a.) is an essential tool of good hygiene and active ventilation for the casted upper or lower limb.

The fifth layer (10a.) is divided by imbedded channels as (12).

Layer (10a.) might have embedded channels (12) in the shape of squares of half by half inch.

The depth of each channel might be one mm. or more.

The embedded channels (12) can be made deep as able to reach layer C and layer and layer B.

Fifth layer (10a.) has domes as (D) to allow air to be stored or circulate in within its empty space.

On the apex of each dome a protrusion (11) helps the layer (VI) from collapsing completely towards layer (V).

The atmospheric air will be stored and exchanged between the empty spaces of the protrusions (11).

The gases, unwanted smells, excessive heat, bacteria, and new viruses escape the immobilizing cast by means of the empty spaces located between protrusions (11).

Under each dome (D) an active ventilation circulates allowing fresh air to reach the wounded skin 3.

Under each dome (D) an exchange of gazes, fresh air, bacteria, new viruses and waves of heat takes place.

The gases, bacteria, viruses and unwanted heat escape from layer (10a) via holes 10.

The gases, bacteria, viruses and unwanted heat travel towards the holes 14 of layer 16.

The fifth layer (10a.) has the hardness in Shore A as 35-40 being similar as the hardness of upper layer of membrane (7).

The above indicated similarity of hardness for fifth layer (10a.) and upper layer of (13) will keep uncompressed room and stored air supply inside all and each dome(s) in (D).

### Sixth Layer

The sixth layer as in (13) is made of a polymer as an EVA™ material or Plastazote™ layer of 0.5 mm thick.

Layer (13) of hardness in Shore A as 35-40 being similar to the hardness of previous fifth layer (10a.).

The similarity of hardness in layer (13) and in layer (10a.) will allow the protrusions (11) to keep the layer (10a.) away from collapsing in between the protrusions (11).

The layer (13) can be porous and especially multiperforated as in (10a.).

The above indicated hardness of layers (IV) and (V) can be modified in accordance to the needs of each wounded skin.

### Seventh Layer

The seventh layer as (15) is an immobilizing layer of fiberglass cast or Plaster of Paris in order to reduce the cost of the cast.

Therefore, the seventh layer (15) can be a combination of three or more casts overlapping each other.

In certain clinics the fiber glass casts are replaced by plaster of Paris casts.

In some fracture clinics cotton is placed optionally between layer (13) and layers of fiberglass casts (15).

Layers (IV), (V) and (VI) contribute in offering a TCC that absorbs the pressure of edema and consequently.

Layers (IV), (V) and (VI) is a ventilated TCC offering a comfortable rehabilitation with less need to recast the injured limb.

It saves time and money to the system by not having to replace the TCC with a larger one more frequently.

The air flow inside the cast will dissipate bad odors and make the cast less itchy and more hygienic by reducing the risk of skin infection or maceration.

This above indicated way of inserting inside a casted limb the fourth layer made of four laminated membranes each with a unique value of compression or hardness is an innovative tool to decrease the negative effects of edema.

This new principle will be used in many designs of immobilizing casts in the near future.

### FIG. 1A Explosive Arrangement of Venting Layers

Inside this FIG. 1A we see an explosive view of layer (VI) supposed to cover layer (V) and we see layer (IV).

Layers (IV), (V) and (VI) are all inserted inside a tubular expandable stockinette.

This FIG. 1A is having its layer (IV), (V) and (VI) similar to the layers of previous. FIG. 1. Inside this FIG. 1A we see bottom layer (Iv) perforated by holes (8a). This layer (IV) is made of four layers from the bottom as layer (A), (B), (C) and (D). Each layer has a different hardness.

The hardness increases from layer (A) to layer (D). These layers absorb gradually the compression force coming from the wounded limb affected by edema.

This layer (IV) is similar as a “shock absorber”. This is because it does not allow the casted limb to feel trapped and tight inside the fiberglass cast.

This layer (IV) has grooves as in 8.

This is in order to add more flexibility to this layer during its installation on the injured leg.

The wholes (8a) allow the bacteria, the bad smells and excessive heat to escape the wounded skin towards the outside area of the immobilizing fiber glass cast.

As layer (V), it is located between the lower layer (IV) and between the upper layer (VI). This layer (V) is also a flexible layer. And its hardness is like the hardness of layer (D) belonging to the quadruple layer (IV).

This layer (V) is an EVA or medical silicone product. It is longitudinal and divided by grooves as in 12.

Layer (V) might be 1.00 mm thick.

It is perforated with wholes of half cm. of diameter as (8a.).

These wholes (8a) allow the bacteria, the bad smells and excessive heat to escape the immobilizing cast and accelerate the healing process of the diabetic wound.

This layer (V) has protrusions 11 to be strong enough not to collapse by any external force coming from the edema skin compression.

These protrusions 11 might be 2.5 mm. tall.

As for membrane (VI) might be an EVA or silicon layer with perforations as in (8a).

The perforations (8a) will make this layer lighter in weight and will make the product more aerated.

This layer (VI) has grooves (8) thus adding extra flexibility to this membrane.

All above indicated layers (IV), (V), and (VI) are inserted inside a an expandable tubular stockinette to represent a venting and compressible orthopedic stripe.

Above indicated membranes (IV), (V) and (VI) are free to move independently inside the tubular stockinette during installation on top of a wounded leg.

### FIG. 1.B Three Venting Stripes for Shin Cover

Inside this FIG. 1B there is an apparatus or venting cover (A,B,C) to be applied on top of a tibia or the shin.

This (A,B,C) cover is considered to be also as a shock absorber against the idema compressive forces generated by the wounded leg.

This cover (A,B,C) has the advantage to be installed and to be adaptable to shapes or curves of any shin horizontally and vertically.

In this case there the need to use a molded or injected or 3D piece is deferred or rejected.

This figure represents venting stripes as A, B and C. Each stripe is sewed to the next one by sewing threads as in 200.

Each stripe A, B and C is externally protected by a tubular expandable stockinette 100.

Stockinettes A, B and C are assembled all together by stitches 200. Rounded portions (400) are sewed to venting stripes A and C by means of stitches 200.

Rounded portions (400) might be connected to stripes A and B also via surgical tapes instead of via stitches 200.

Inside each of segments A, B and C are incorporated from the bottom the multi hardness layer (IV), followed by a bumpy layer (V) the bumps are not visible inside this figure.

On top of the bumpy layer (IV) there is an upper flat and mufti perforated layer as (VI).

All these arrangements and descriptions of these above indicated layers are described in an analytical form inside the previous figure of FIG. 1.A.

### FIG. 1.0 Two Venting Stripe for Achille's Tendon and Calf Cover

Inside this FIG. 1.0 there is a calf cover 500. Three bold arrows indicate the direction to of how this cover 500 will be used on top of the dorsal side of the leg 600.

This leg 600 has a wounded zone as 3 to be covered by a wound dressing 4.

The phalanges are externally protected by protective layer 5.

A calf “ventilating cover” as 500 is adaptable smoothly to the curved shape of calf 700.

This “ventilating cover” 500 might be anchored on the dorsal zone of the foot 600 by means of surgical tape as one piece to be installed around the ankle at (k) and a second piece around the curved calf at (L) plus a last and third stripe of surgical tape at the opposite end of the ankle at (J).

The calf cover 500 might be made of longitudinal venting tubular stripes made of stockinette as (A) and as (B).

Longitudinal stripes (A) and (B) are connected by stitches 200.

Stripes (A) and (B) are bend towards each other and cover all together the calf with Achille's tendon.

The tubular stockinettes 100 contain four multi perforated membranes of EVA or plastazote™ or medical silicone as described in next FIG. 1D.

### FIG. 1.D

Cross section of venting stripes for calf and Achille's tendon as 1000

This FIG. 1.D is a cross section of the calf cover 500 located inside the previous FIG. 1C.

Side (A) and side (B) are connected by stitch 200. Each side (A) and (B) is made of expandable stockinette 100.

Side (A) and side (B) are externally attached by a stripe of surgical tape around the leg's calf.

Inside the stockinettes of side (A) and (B) there are four layers made of EVA or Plastazote™ or medical silicone.

From the bottom there might be layer (IV) to be made of four different membranes as (a, b, c, d).

Layer (IV) becomes compressed as soon as the wounded leg becomes swollen by the edema force.

Layer (IV) is protecting the wounded leg from blockage of blood vessels.

Layer (IV) has perforations 17. On top of layer (IV) lies down a protruded membrane as layer (V).

Layer (V) has holes as 17.

On top of protruded layer (V) lies a flat layer of EVA or Plastazote™ or medical silicone as layer (VI).

Layer (VI) has perforations as 17 as well.

Layer (IV) has grooves as 800 to allow it to be more flexible during its installation around the calf and the Achille's zone.

Grooves 800 might be present by choice on protruded layer (V).

Optionally groove 800 might be present on flat layer (VI).

### FIG. 2A (Diabetic Foot Ulcer)

Inside this figure it is shown an injured foot at 3 and covered by a tubular stockinette as (1).

The stockinette (6) is covering partially the phalanges as in (2).

Under the foot as in location 3 there is a neuropathic wound or (DFU) diabetic disability foot ulcer.

Over the wound there is a medicated wound dressing as 4.

The phalanges are covered by external shield 5.

### FIG. 2B (Venting Cover for the Shin and Phalanges)

Inside this figure there is cover (1) made of EVA™ or medical silicone or Plastazote™ thermoformed or injected layer.

It covers the tibia shin/bone the metatarsals and the phalanges of the injured leg.

At the near end bottom there are two ear shaped portions as (2) also made of EVA™ or Plastazote™.

Cover (1) can be made of any polymer or silicone gel material.

It is made with engraved channels as in (3).

Each channel (3) can be deep as much as one to one and half mm. and wide as one to one and half mm.

Cover (1) has holes (4) of one to one and a half mm. diam.

The engraved channels (3) can be spaced by a distance of ten to fifteen mm.

The channels (3) allow cover (1) to bend with greater flexibility upon installation on top of the shin and the metatarsals.

This cover 1 might be made by three separated longitudinal portions.

Each portion might be inserted inside a tubular stockinette.

The three tubular stockinettes are sewed together to form a single piece.

Cover (1) is made of three to four laminated layers.

The four layers do not exceed the total thickness of three to four mm.

There are the following layers as (A), (B), (C) and (D).

Holes (4) cross the cover (1) from layer (A) to layer (0).

Channels (3) can represent any geometric practical shape as rectangles, squares, lozenges, ovals, circles.

The ear shaped portions (3) are slightly domed in order to cover the malleoli in a comfortable fashion.

Portion (3) is useful to cover both malleoli.

rounded or oval portions (3) are attached to each other by a surgical tape or a thin and soft chord as in (5) at proximity of Achille's tendon.

Cover (1) starting from its bottom layer as (A) is the softest layer compared to layers (B), (C) and (D).

Hardness of bottom layer (A) in Shore A is 20 to 25. Is in direct contact with stockinette (1) as in FIG. 2.A.

Hardness of layer (B) in Shore A is 25 to 30.

Hardness of layer (C) in Shore A is 30 to 35.

Hardness of layer (D) in Shore A is 35 to 40.

Cover (1) with its membranes (A), (B), (C) and (D) represents a compressible and unique tool capable to store the edema compressible force generated by the swollen limb.

The membranes (A), (B), (C) and (D) will not transfer the compressible force coming from the swollen limb backwards around the swollen limb affected by edema.

This mechanism takes place because cover (1) has the adequate softness or hardness to absorb the compressive force of edema without resisting it.

The compression force of edema is stored inside cover (1) between the wounded limb and the internal walls of the immobilizing fiber glass cast.

The compressible cover (1) will decrease the asphyxiating and persistent pain that takes place in and outside the wounded limb.

By using cover (1) it eliminates the need to replace the first immobilizing fiber glass cast more frequently.

Reduced sensation of edema means faster recovery and having more productive patients.

Optionally, cover (1) can be made only by one single layer representing the acquisition of a compressible force able to equal the edema's compressible force.

In this case cover (1) will be manufactured in a vast range of thicknesses and consequently made of various hardness as in Shore A.

This diversification of thickness and hardness will apply differently in accordance to the health and magnitude of each wound or injury.

### FIG. 2.B1 (Three Separated Venting Stripes for the Shin)

This Figure of 2.B1 is the same as FIG. 2.B the FIG. 2.B is an object made of one whole piece but inside this Figure of 2.B1 we have the three segments as A, B, and C. All segments are the same having engraved channels as 3 and being perforated as wholes 4.

Each segment A, B, C and D is made of four layers.

These segments have the same features of the element shown inside FIG. 1.

Each segment of A, B, C is inserted inside an expandable stockinette as inside stokinette 20, 30, and 40.

The three stockinettes 20, 30, and 40 might be sawed together and create from the three pieces one whole piece similar to the piece of FIG. 2.B.

This arrangement will make the manufacturing procedure more easy because the tubular stockinette 20, 30, and 40 might be more easily adapted to the curves of the shin/tibia of the leg.

In this design the above indicated piece 1 can be adapted to any shape.

But by using the part of FIG. 2.B because there is not tubular stockinette we must use a molding or ejecting procedure or a 3D fabrication technology.

At least for now all these technologies are available, but they might be time consuming and expensive.

### FIG. 2.B2 (Complete Venting Frontal Shield for the Shin)

Inside this figure of 2.B2 we can see the wounded leg zone at 3 being covered by the tubular stockinette 1. covered by perforated layer (C) and installed via surgical tape (19 and by chord 5.

Inside element (III) is exposed a tubular stockinette as 1.

A protective cover 5 shields the phalanges and the wounded zone 3.

A wounded zone 3 is under the phalanges.

A perforated layer C covers the tibia/shin.

A surgical tape (19 anchors cover C to the tibia/shin.

A chord 15 anchors the device on top of the malleoli.

Concave pair of discs 6 cover the ankle of the foot.

Layer C might have holes 4 all over its surface of diameter of half to two mm.

Cover C has horizontal and vertical channels 13 criss crossing each other. Cover C might be made of three or four layers as A, B, C and D.

Each layer might have a unique value of hardness depending on the patient's health.

Layer A has the least hardness and D has the highest hardness.

This above indicated design allows the skin to be compressed by the edema force and this compressive force of edema will not be transmitted to the immobilized leg or either to internal walls of the fiberglass cast.

This above indicated perforated layer C of lower level (III) might be placed under cover (II).

Cover (II) has spaced protrusions 11.

The spaced protrusions 11 might be one to two mm. tall.

Cover (II) is perforated by holes 4.

Each hole is half mm. of diameter.

The diameter of each hole might change in accordance of the situation of each patient.

This frontal shin's shield (II) is made as one thermoformed piece.

It can be made by extrusion or compression molding or 3D technology.

The same applies for manufacturing layers (I) and layers (III).

Cover (III) is as a “shock absorber”.

Cover (III) stores/absorbs the edema force potential energy that is originated from the swollen leg.

Cover (II) is an instrument of distributed ventilation by means of the spaced protrusions 11.

Cover (II) is a mean to transfer bacteria, heat and smell away from the casted zone of the wounded limb by means of the hole as in 4.

Layers (I), (II) and (III) might be prefabricated or custom made.

These above indicated layers are anchored to the injured leg separately or all together by means of surgical tapes as in (1^{st}).

They might be anchored to the malleoli by rounded or oval discs as 6 via chord 5.

Layer (I) made of EVA, or Plastazote™, or medical silicone. It is soft and flexible easy to be shaped on the curved tibia/shin.

It has grooves 13 to allow more flexibility when anchored on top of protruded layer (II).

### FIG. 2D.A

(Pathway of bacteria through a shock absorber and through a protruded layer)

This Figure of 2D.A is a cross section view of a shock absorber membrane mounted under of a protruded membrane as in (2).

Layers 1,2,3 and 4 are laminated to each other as demonstrated in previous figures as FIG. 1 and as in FIG. 1A.

Layers 1,2,3 and 4 represent a “shock absorber” membrane as (C).

Protruded layer (2) lies on top of the shock absorber layer (C).

Bacteria (B) travel through the holes (H) of the membrane (C).

Bacteria (B) travel through the holes H and get dissipated between the empty spaces of the domes (D).

Bacteria (B) exit the immobilizing fiberglass cast from the upper open end of the fiberglass cast near the knee by means of existing holes as D located on walls of dome (D).

Air as (A) enters inside the protruded layer (2) and it travels between the protrusions P then enters inside the wounded area of the casted leg via holes (H).

The thickness of layer (C) might be one to three mm.

Layer (C) might be adequately hard for not collapsing by the edema compressive force of the wounded limb.

### FIG. 2D.B (Dorsal Assembly of Shock Absorber Device)

Inside this Figure of 2D.B the wounded leg L is reversed.

Cover C1 is ready to protect the area of the calf and the tendon.

The leg L has protruded layer (2) anchored via chord 9 at malleoli 3 and via surgical tape (2ST).

Leg L is covered by tubular stockinette 200 and protected by at the toes by cover 100.

Cover C.1 is a shock absorber membrane.

C.1 is the first layer to cover the calf and the Achille's tendon.

C.1 has grooves as in 3 and holes as in (R).

### FIG. 2D.C (Dorsal Assembly of Protruded Device)

Inside this FIG. 2D-C leg (L) is being repositioned and ready to be covered by protruded cover (C.2).

Cover (C1) to be in direct contact with tubular stockinette as in 1.

Cover (C.1) represents the similar 4^{th}. Layer present inside FIG. 1. It acts as a shock absorber against the edema forece.

Inside this figure as FIG. 2D-C it has perforations (P) of diameter of 1 to 3 mm.

It is made of a thermoformable polymer as an EVA™ or Plastazote™ material.

It can be made of medical silicone or gel.

It has imbedded channels as in (3).

The imbedded channels (3) can form different shapes as rectangles (R) or squares or lozenges or oval or round circles.

The presence of channels (3) make this cover C1 more flexible when installed on top of calf (200).

This cover C1 is made of four laminated layers as described below.

First inner layer (invisible inside this figure) as (A) has hardness as Shore A of 20 to 25.

Layer (A) directly close to the tubular stockinette 1.

### FIG. 2D.a (Pathway of Bacteria and Gazes Protruded Layer and Inside the Shock Absorber Layers)

Inside this figure we can see in a magnified fashion the cross section as cover (2).

We can spot round shaped balls or spheres as in (s) and as in (b) escaping the wounded limb towards the outer space away from the fiber glass cast.

The balls or spheres (s) and (b) are particles of bacteria or molecules of gazes from sweat or oils or pus.

It is obvious that spheres or particles (s) and (b) travel through layers (c) and layer (2) via the holes (H).

Particles of atmospheric air labelled as units (A) travel in and out through layers layer (2) through layers (1, 2, 3 and 4). Layers (1, 2, 3 and 4) represent layer (C).

Particles of air (A) enter inside the immobilizing cast by means of the open aperture of the fiberglass cast near the knee of the injured leg.

Particles of atmospheric air (A) enter around the wounded zone via the porous walls of the fiberglass cast layers.

Particles (A) move randomly between protrusions P and inside the domes (0) until they reach the area of the wounded limb.

The domes (D) are with height of up 2 to 3 mm.

Particle (A) kicks out from inside the cast harmful bacteria, unwanted smells and excessive heat.

Layer (C) is the main tool that absorbs the edema force or pressure by being made of four compressible layers.

Layer (C) transfers the bacteria from the wounded area towards the outer sides from the fiberglass cast.

As layer 1 of layer (C) is with least resistance to edema pressure.

And as layer 2 of layer (C) is with higher resistance to edema compressing pressure.

And as layer 3 of layer (C) is with higher resistance to edema pressure as compared to layer 2.

And as layer 4 of layer (C) is with higher resistance to edema pressure compared to previous layers 1,2 and 3.

### FIG. 2D-B

This Figure is showing protruded layer (2) being installed on top of hidden layer (C).

Tubular stockinette 1 covers leg (L).

Protruded layer (2) is attached to leg (L) by means of surgical tape (2.ST).

Protruded layer (2) is anchored to malleoli (3) via surgical tape or a knotted chord as in (C).

Phalanges are covered by upper and lower sides by cover (100).

### FIG. 2.D-C

Inside FIG. 2.D-C the leg (L) is being repositioned and ready to be covered by (C.1).

Cover (C.1) be in direct contact with tubular stockinette as in 1.

Cover (CA) represents the similar 4^{th} layer present inside FIG. 1.

Inside this figure as FIG. 2.D-C it has perforations (P) of diameter of 1 to 3 mm.

It is made of a thermoformable polymer as an EVA™ or Plastazote™ material.

It can be made of medical silicone or gel.

It has imbedded channels as in (3).

The imbedded channels (3) can form different shapes as rectangles (R) or squares or lozenges oval circles or round circles.

Presence of channels (3) make this cover C1 more flexible when installed on top of calf (200).

This cover C.1 is made of four laminated layers as described below:

First inner layer as (A) has hardness as Shore A of 20 to 25. Layer (A) directly close to the tubular stockinette 1.

Second layer as (B) has hardness as Shore A of 25 to 30.

Third layer as (C) has hardness as Shore A of 30 to 35.

Fourth (visible) layer as (b) has hardness as Shore A of 35 to 40.

This cover C.1 has the function to be compressed by the edema force coming from the wounded leg (L) for an improved rehabilitation during the immobilization phase by the fiber glass cast.

### FIG. 2D-D

Inside this FIG. 2D-D a wounded leg as (1) is covered at the phalanges by protective cover (100).

The posterior part of (L) is covered along the calf and along the Achille's tendon area by protruded ventilating cover as (C2).

The frontal part of leg (1) is covered by protruded shield as layer (2).

Layer (2) covers the shin and the phalanges portion.

Layer (2) and has protrusions 901.

Layer (C2) have protrusions as 901.

Layer (2) and layer (C2) have holes as 902.

Layer (2) and layer (C2) have engraved channels as 900.

Layer (2) and layer (C2) are installed on leg (1) by surgical tapes as in 700A, 700B and 700C.

### FIG. 2D-E

Inside this FIG. 2D-E injured leg (1) is immobilized by fiberglass cast 1000 and by fiberglass cast 1050.

The injured leg (L) might be contained by external cover 2000A.

2000A shields the dorsal part of the leg.

The injured leg (L) might be contained by external cover 2000B.

External shield 2000B protects the shin of injured leg (L).

Cover 2000A and cover 2000B have holes 903.

Covers 2000A and 2000B have grooves 901.

Grooves 901 add more flexibility to covers 2000A and to cover 2000B.

Grooves 901 aid for a smoother installation around the leg (L).

Holes 903 allow the escape of bacteria and smell from the pores of the fiberglass cast.

Bacteria, smells and heat escapes the casted limb from the upper end of the total contact cast at 600.

Bacteria, unwanted smells and heat are surfing between the protrusions 901 of layer (C2) and layer (2).

Bacteria, unwanted smells and heat escape the fiberglass cast as shown by directions of arrows 600.

### FIG. 2D-F

Inside this FIG. 2D-F all the layers are similar as compared to the layers of FIG. 1 except layer (IV).

First layer (I):

The skin of an immobilizes limb is shown as (1) being affected by edema force as indicated by arrows (2).

The direction of the edema pressure (2) against the internal walls of immobilizing cast (15) is shown by arrows (2). The wound is in (3) covered by a medical wound dressing as in (3b.). The bacteria and different sources of vapours as (9) exit from the surface of the casted skin (1) towards and underneath the open sides of the cast 16

Second layer (II):

The limb (1) is optionally covered by a sterilized and porous cotton layer as in (5).

Third layer (Ill):

The whole limb (1) is covered by a surgical hydrophilic sterilized expandable tubular stockinette (6).

Fourth layer (IV): or indicated as layer 7 An EVA™ or Plastazote™ layer as 7.

Might be with thickness from to 7 mm.

Might be with hardness of shore A as durometer 20 to 25.

As edema force increases layer 7 might gradually decrease from its original thickness of 7 mm and become up to 1.5 mm thick.

The compressive force of edema became stored inside layer 7.

Layer 7 is a storage of potential energy by being compressed by edema.

to around 3 mm. as soon as the edema force will compress it from inside the wounded zone towards the internal walls of the fiber glass cast.

The thickness of compressible layer 7 will be decided in accordance to the health of the patient.

Layer 7 might be made of durometer A less than 20.

The thickness of layer 7 might be needed to be more than 7 mm if the Edema is making the swollen limb excessively big.

Layer 7 has engraved channels as 8 to add additional flexibility.

Layer 7 has holes as 8a.

Holes 8a. are a mean of ventilation for the internal surface of the cast

Holes 8a. are a mean to let bacteria, excessive heat and unwanted smells to escape the wounded zone.

Holes 8a. are a mean to make this membrane lighter.

Fifth layer (V):

Fifth layer (V) is labelled also as (10.a).

It has same durometer as layer 7 of A 20.

Layer 7 has domes D.

Domes D receive fresh atmospheric air from its upper opposite side.

Domes D have holes 10 for exchange of gazes, heat.

Through holes 10 bacteria escape the cast.

Through holed 10 unwanted smells escape the cast.

Layer (10.a) has protrusions able to withstand the edema pressure coming from the wounded leg.

Bacteria and vapours as (9) escape from the wounded area as (3) until they are expelled outside from the cast by means of protruded layer 10a.

Atmospheric air indicted as 1000 enters automatically cast 15 and ventilates layers (VII), (VI), (V), (IV), (III), (II) and the wounded area as in (3).

### FIG. 2D-G

This figure as 700 is a perspective view of a total contact cast inside a Rocker bottom cast shoe as 150.

The total contact cast (TCC) is shown as 146.

The total contact cast (TCC) 146 might cover entirely the toes as in 143.

The total contact cast (TCC) 146 with the Rocker shoe cast 150 alleviate mechanical load on existing foot ulcer.

The total contact cast (TCC) 146 might allow fresh atmospheric air as in 143.B to ventilate the immobilized lower leg.

The atmospheric air as in 143.B might allow the bacteria with excessive heat and unwanted gaseous smells to escape from the internal walls of the total contact cast (TCC) 146.

The atmospheric air as in 143.B might alleviate the side effects of edema around and over the wounded lower leg.

### FIG. 2D-H

This figure as 701 is a perspective view of a total contact cast inside a Rocker bottom cast shoe as 150.

The total contact cast (TCC) 146 might be open at 142,

The aperture 142 of the total contact cast (TCC) might allow the casted skin to be ventilated with fresh air.

The aperture 142 of the total contact cast (TCC) might allow excessive heat, bacteria and unwanted odors to escape the internal walls of the total contact cast 146.

The fresh air as 142.A might alleviate the pain from the edema exercised all over the wounded lower leg.

FIG. 2D-I (Birth of Edema from a to B)

Inside this figure it is shown the protruded layer as in 10.

Layer 10 has holes 11.

Layer 10 has domes 12.

Layer 10 is not compressible.

Layer 10 allows air bacteria and heat to circulate inside the domes 12.

Layer 10 allows air bacteria and heat to circulate between the protrusions at 13.

Zone A represents the skin of a wounded lower leg before edema starts taking place.

Zone B represents the skin of a wounded lower leg after edema took place.

Zone B became bigger or swollen than zone A because of occurrence of edema.

On top and under the zone A lies uncompressed layer 14

On top and under the zone B lies compressed layer 14.

Layers 14 have perforations 15.

Perforations 15 allow air to penetrate the swollen lower leg area.

Perforations 15 allow gazes and pathogens to escape the wounded area of the lower leg.

The wounded lower leg as in A and as in B is immobilized by two or three layers of fiberglass cast.

As the edema takes place from A to B the layer 14, if compared inside zone A versus zone B becomes thinner in zone B.

Layer 14 is an absorptive weapon of edema allowing the blood vessels to maintain a normal circulation.

Layer 14 absorbs the edema pressure without forcing the care giver to change the immobilizing fiberglass cast by a larger one.

Layer 14 is a shock absorber for edema.

Layer 14 is a mean of generous ventilation for the wounded zone B.

Layer 14 is a mean to accelerate the healing of the wounded area A and B.

Layer 14 is made of one to four laminated layers.

Layer 14 has its inner layer close to the wounded skin as A.

Layer 14 has inner layer A being softer than layer B.

Layer 14 has layer B being softer than layer C.

Layer 14 has layer C being softer than layer D.

Layer 14 has layer A with hardness of Shore A 20 to 25.

Layer 14 has layer B with hardness of Shore A 25 to 30.

Layer 14 has layer C with hardness of Shore A 30 to 35.

Layer 14 has layer D with hardness of Shore A 35 to 40.

Layer 14 might be made of one layer of total thickness of 4 to 8 millimeters.

Layer 14 might be made of hardness of Shore A 25 to 30 being able to absorb fully the compressive force of edema.

Layer 14 has embedded grooves as 16 over its outer layer D in order to enhance the flexibility of the device.

Layer 14 will preserve its ventilating traffic when fully compressed by the edema squeezing force as shown in area of A and B.

Layer 14 will preserve its ventilating traffic function when fully compressed by the edema squeezing force as shown in area B wounded lower leg area of wounded lower leg in A.

The protruded layer 10 is fully covered by layer 17,

Layer 17 might have perforations 18 of diameter half millimeter to make the device lighter and increase the aeration between the wounded skin and the pores of 19 of the fiber glass cast 16.

The immobilizing fiberglass cast 2616 might be replaced by polyester fabric spacers.

Plaster of Paris.

### FIG. 2D-J

With fabric spacers: A. before edema B. after edema inside this Figure as 2D-J there is a casted lower leg as A and B.

Inside A the wounded limb is freshly casted by plaster of Paris or by fiberglass.

The casts 26 might be two to three layers on top of each other.

The lower leg is swollen by edema as shown inside B.

A compressible layer 14 is shown before edema takes place as inside figure A.

Layer 14 is compressed by force of edema as shown in figure B.

Fabric spacers 11 is not compressible.

Fabric spacer 11 optionally might be compressible and share the function of compressible layer 14.

Layers 14,11 and 17 are perforated to allow more ventilation inside the total contact cast 26.

### FIG. 2D-K

Inside this FIG. 2D-K there are two pictures of tubinettes as it follows:

(I) There are three longitudinal tubular tubinettes as in (1) sawed together by stitches as in (1).

(II) There is one opened tubinette as in (T). With an extracted venting system made of upper layer 1, made of middle layer 3 and bottom layer as 2.

These above indicated layers allow the wounded skin to be ventilated. As expressed inside my previous patent application PCT/CA2028/051668.

This tubinette (T) is easily applied on curved surfaces as on calves and this is because of the presence of grooves as in 5 on layers 1,3 and 2.

This tubinette (T) is a good source of aeration to the skin because of the presence of perforations as in 4 on top of layers 1,2 and 3 (not visible). The atmospheric air circulates between the protrusions 6 and ventilates the wounded leg.

### FIG. 2D-L

Inside FIG. 2D-L there is a lower leg 1 protected by a sock 2.

The shin as in 3 and the calf as in 4 are covered by a venting system.

The venting system is made of an external tubinette as in 1a. 1b. and 1c.

The tubinettes 1a., 1b. and 1c are anchored to the leg 1 by means of Velcro attachments as in 5, 6 and 7.

The tubinettes 1a., 1b. and 1c. allow air to penetrate the wounded skin of leg 1.

The total contact cast treatment might be terminated, and the wounded lower leg might be inserted inside a removable rocker walking cast or boot.

Walking braces causes the presence of excessive heat, itchiness, bad odours, bacteria, pain accompanied by discomfort because the wounded limb is surrounded by a pad made of foam in tight position.

This figure shows the wounded lower leg before being swollen by edema as in A and after being swollen as in B having a venting membrane made of perforated fabric spacers as in 11.

Inside zone A and inside zone B lies a compressible layer 14 representing the same chemical and physical features of FIG. 2D-I.

Inside zone A and inside zone B lies a perforated layer as

In 17 having the same features of layer 17 present in FIG. 2D-I.

The total contact cast of this 2D-J figure helps the patient to fight the edema pressure as shown in previous FIG. 2D-I.

The lower leg A or B is casted by fiber glass or by plaster. of Paris as in 26.

### FIG. 2D-K

Inside this 2D-K figure there is a tubular tubinette as in “T”. It is shown in I, II and in III.

Inside figure (I) there are three tubinettes connected together by stitches as in 1.

Inside figure (II) there is tubinette “T” showing a protruded longitudinal layer as in 2 with holes as in 3.

On top of layer 2 lies a flat layer as in 4.

On layer 4 there are perforations as in 5.

Tubinette “T” is easily adaptable on any curved portion of the leg because layer 2 and layer 4 are free to move longitudinally inside the tubular tubinette “T”.

Inside figure (III) there is the layer 2 with the layer 4 pulled out from tubinette “T”.

Layer 4 has grooves 6 in order to add more flexibility to the device when anchored on top of a calf.

Protruded layer 2 has grooves 7 to make the device lighter and more adaptable to any curves of the wounded lower leg.

To avoid the disadvantages of removable rocker walking cast supposed to heal diabetic foot ulcer. DFU, it is reasonably concluded that the wounded lower leg must be aerated by the above indicated, venting system as in 1a., 1b. and 1c.

The wounded leg as in 1 might be allowed to receive an adequate ventilation by being protected by venting systems of tubinettes 1a., 1b., and 1c.

The venting systems 1a., 1b. and 1c. can be washed as needed.

The venting systems 1a., 1b. and 1c can be cut to the desired length.

The venting systems can be made of tubinettes of larger or smaller diameters.

### FIG. 2D-M

Inside this FIG. 2D-M there is the lower injured leg 1 by a diabetic foot ulcer being covered by vented tubinettes as in 1a., 1b. and 1c.

By observing at part (I) there is a frontal view of portion T of the vented tubinettes.

By observing at part (II) there is the vented layers 100 as being partially extracted from the venting tubinette of 1a.

By observing at part (III) there is the vented layers 100 as being Partially extracted from the venting tubinett of 1c.

### FIG. 2D-N

Inside this FIG. 2D-N there is a cross section of a removable diabetic walking brace as 50 extremely popular in the orthopedic industries with its rigid plastic walls as 1.

It has its frontal plastic shield as 2.

It has an outer rocker stepping sole as 3.

It has an internal sole as 4 with Shore A hardness as 20 to 30.

It will protect the injured limb of FIG. 2D-L against DFU disease as a classical solution by immobilizing the lower leg by means of its rigid walls as in 1.

The removable walking brace 50 is designed to offload the diabetic foot ulcer (DFU).

The injured lower leg as a general practice is first inserted inside a cushion made of foam as a liner as indicated in 5.

### FIG. 2D-O

The injured lower leg is contained inside a walking brace as 50.

The walking brace has an external bottom stepping sole as 3 with rocker styled shape.

The walking brace is removable via its frontal shield 2. The frontal shield is attached to the injured leg via Velcro stripes.

The walking brace has an internal sole as 4.

The wounded lower leg by diabetic foot ulcer DFU before being inserted inside the walking brace 50 the surgeon or the care giver envelops it inside a foam cushion as 25.

The wounded lower leg by diabetic foot ulcer DFU before being enveloped by the foam cushion as 5 it is contained inside a cotton sock liner.

The wounded lower leg by diabetic foot ulcer DFU after being contained by the sock liner 2 the care giver will attach to the leg the “T” tubular venting tubinettes as 1a. and 1b. on top of the frontal side of the leg and a “T” venting tubular tubinette as 1c. on the back on top of the calf and Achille's tendon.

The attachment of the “T”s venting tubinettes takes place via a mechanical system with Velcro™ or via surgical tapes.

The venting system of the “T”s tubinettes allows the wounded leg by diabetic foot ulcer DFU to heal by receiving a generous ventilation by expelling unwanted odors and excessive heat by kicking out harmful bacteria.

By using the walking brace alone without the “T”s tubinettes venting system the foam cushion 5 would not create a ventilated for the wounded leg by DFU.

### BRIEF DESCRIPTION OF THE DRAWINGS

FIG. 1. shows a diagram of casted skin by fiberglass cast being affected by edema pressure.

FIG. 1A. shows an explosive view of the edema absorptive layer with protruded layer.

FIG. 1B. shows A, B and C tubular stockinette venting units sewed together as one single piece.

FIG. 1C. shows two venting or edema absorptive stripes inside tubinette as A and B.

FIG. 1D. shows a cross section view of venting stockinette A and B covering the calf.

FIG. 2A. shows a patient foot with diabetic foot ulcer.

FIG. 2B. shows a venting cover for the shin having absorptive edema layers as (A, B, C and D).

FIG. 2B1. shows tubinette A, B and C each holding a series of edema absorbing layers.

FIG. 2B2. An explosive view of the shin frontal cover as external covers as (I), (II) and (III).

FIG. 2D.A shows the pathway of bacteria through a shock absorber and through a protruded layer.

FIG. 2D.B shows a venting protruded layer mounted on the shin and it shows an edema reducer layer ready to be installed on top of the calf.

FIG. 2D.C shows a protruded venting layer ready to cover the calf and Achille's tendon area.

FIG. 2D.D shows a leg with a wound cover at the toes and having at the calf plus tibia zones a protruded ventilating cover.

FIG. 2D.E shows a total contact cast covering a wounded leg.

FIG. 2D.F shows a diagram of casted skin by fiberglass cast being affected by edema and protected by layer (VI) having a mono hardness durometer (A).

FIG. 2D.G Cast walking shoe with closed total contact cast.

FIG. 2D.H Cast walking shoe with open end of total contact cast.

FIG. 2D.I Wounded lower leg before and after edema with Protruded ventilating membrane.

FIG. 2D.J Wounded lower leg before and after edema with venting fabric spacers.

FIG. 2D.K (I) Three attached tubinettes
(II) Single tubinette with pulled out venting layers

FIG. 2D.L Lower leg with venting tubinettes.

FIG. 2D.M Lower leg with venting tubinettes with pulled out venting layers.

FIG. 2D.N Removable rocker walking brace with removable leg's protective cushion.

FIG. 2D.O Lower leg inside walking brace after having a total contact cast with venting tubinettes.

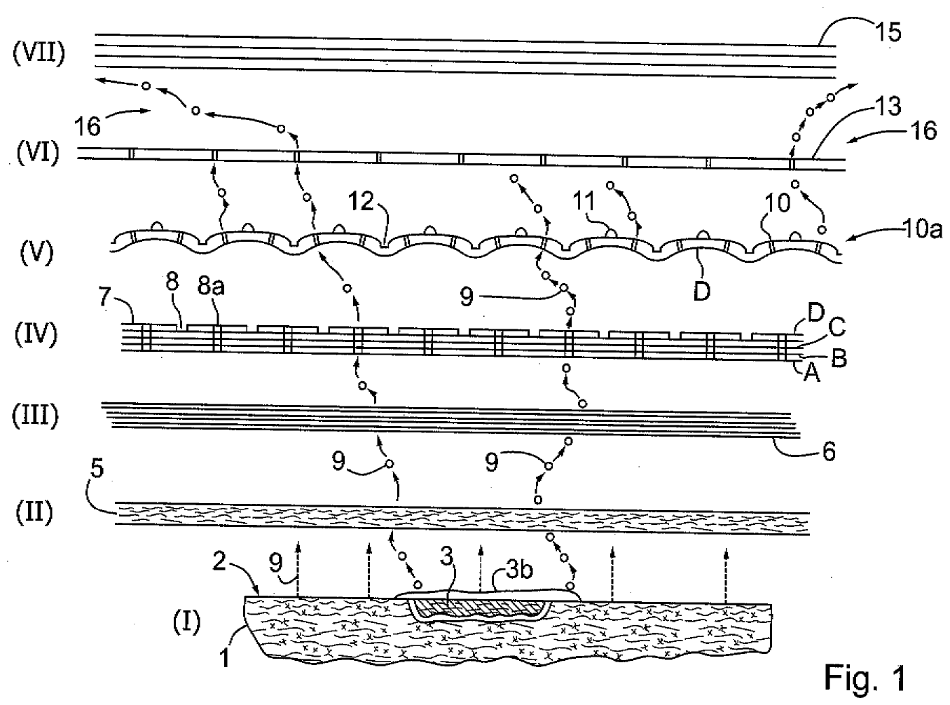

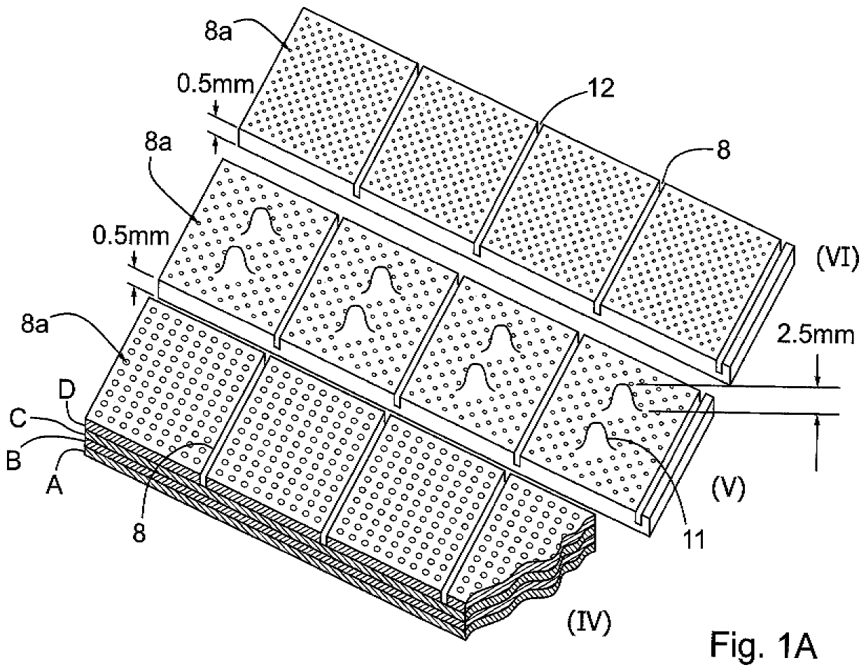

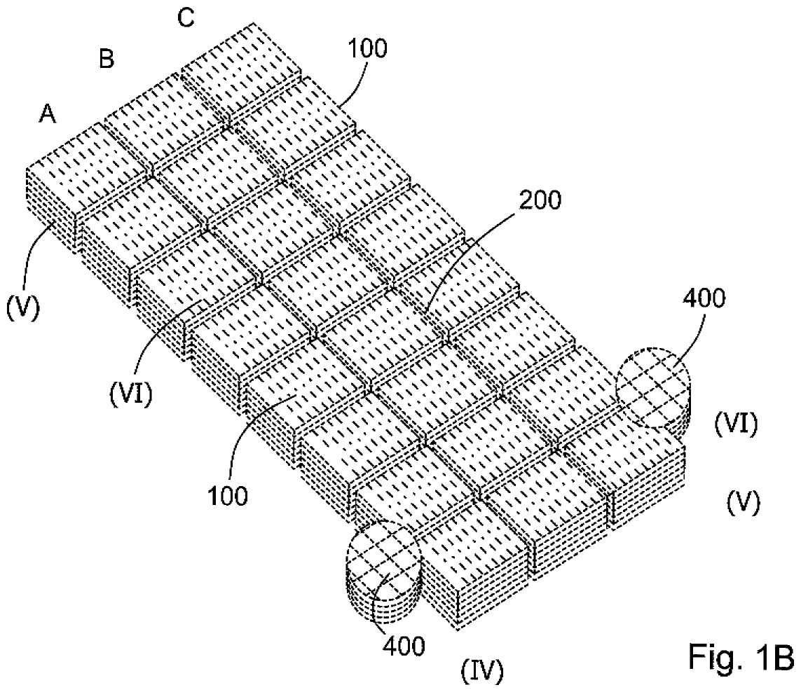

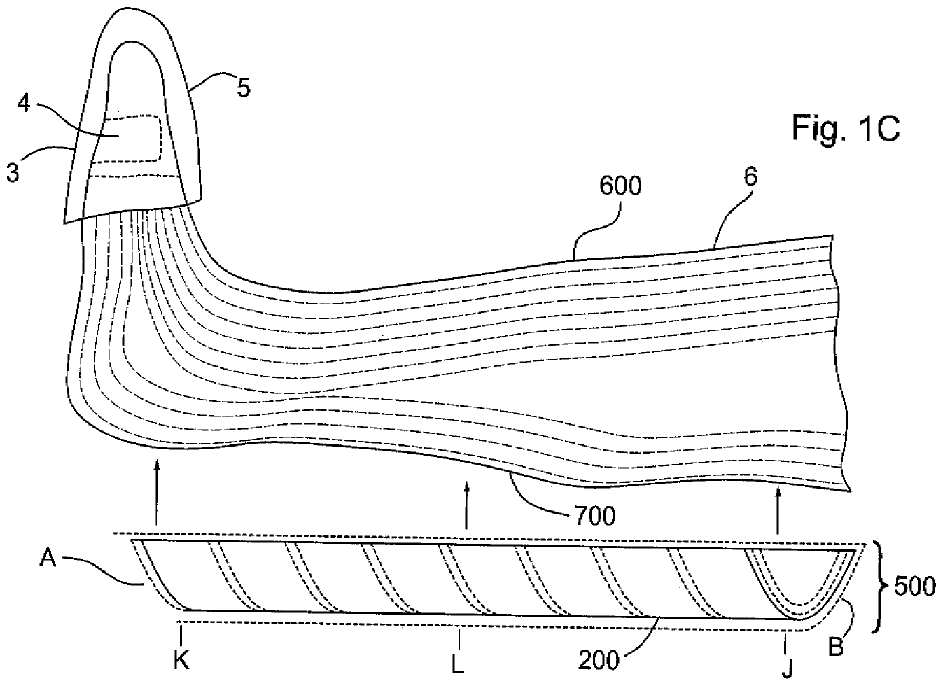

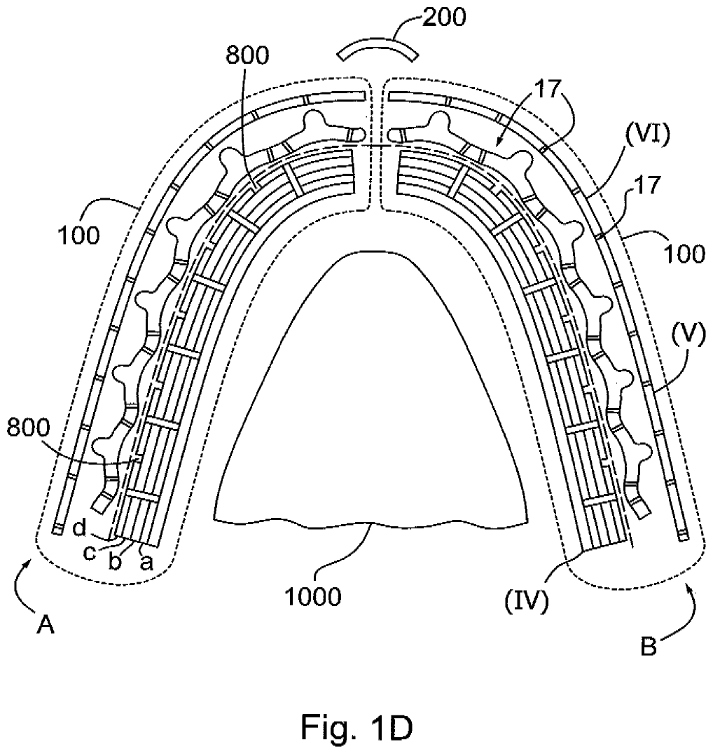

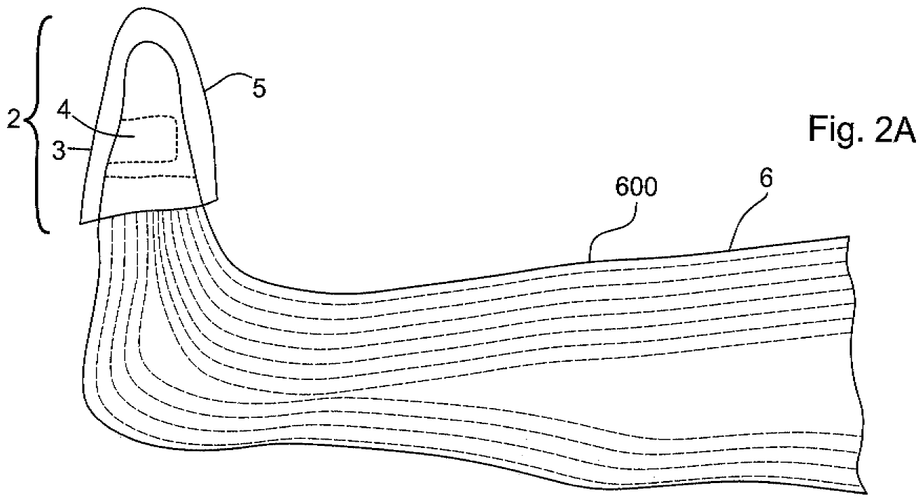

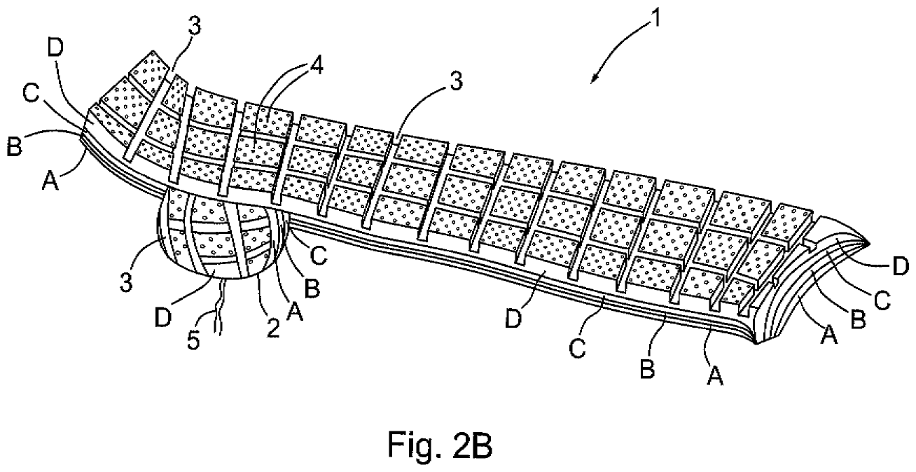

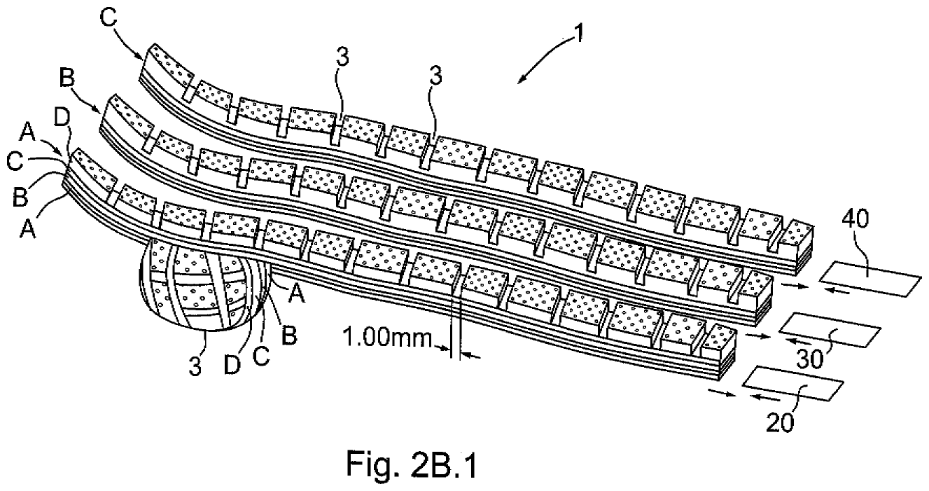

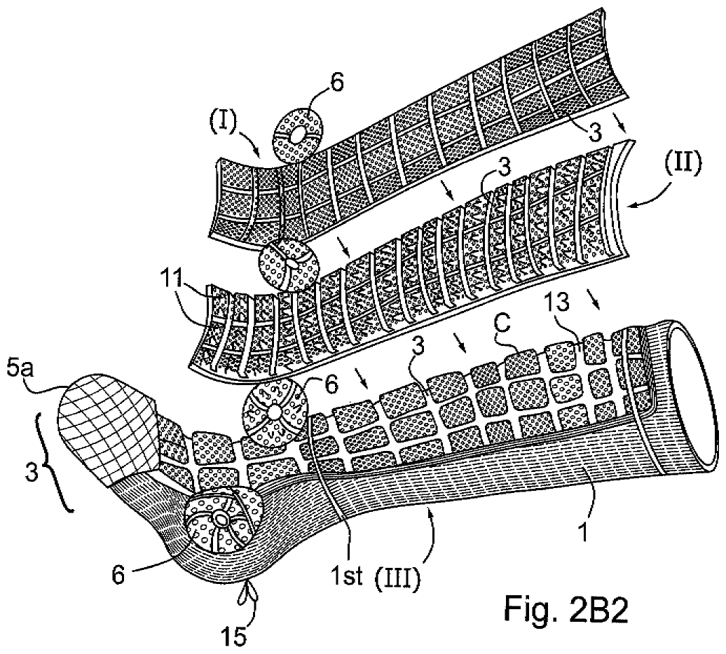

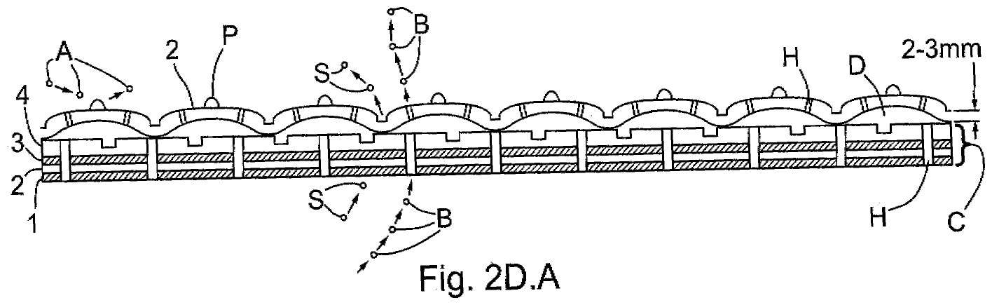

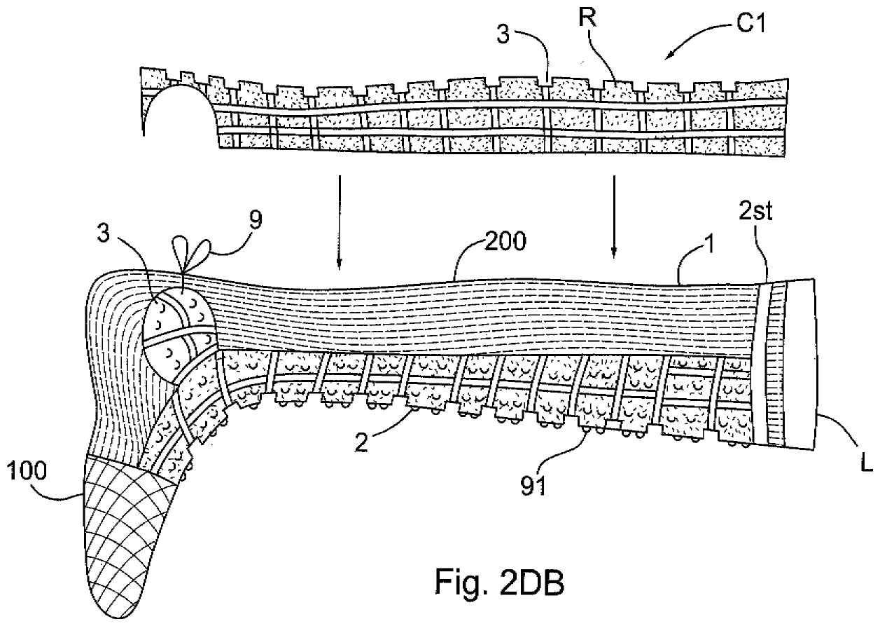

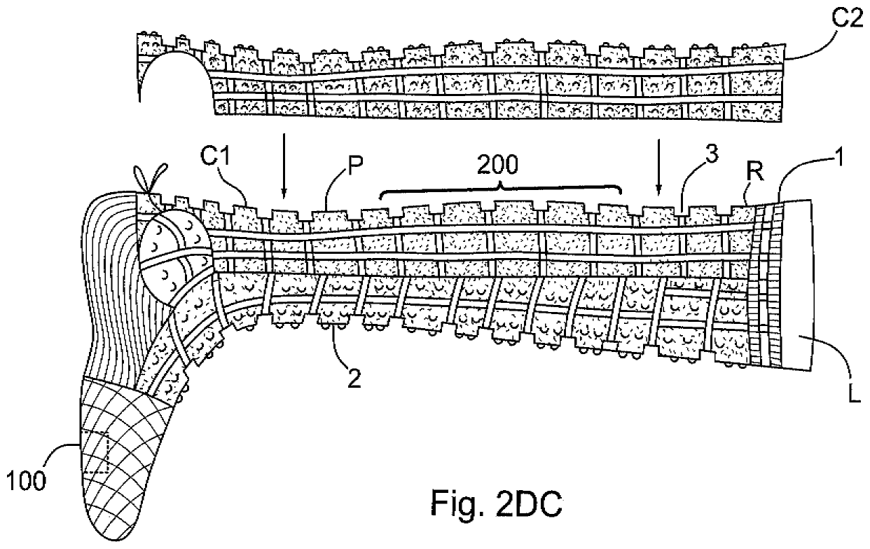

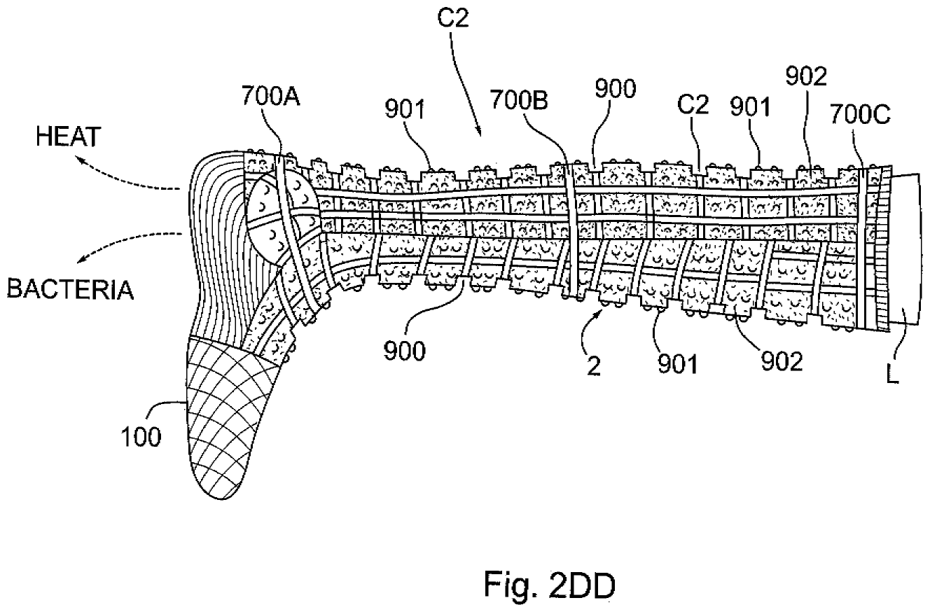

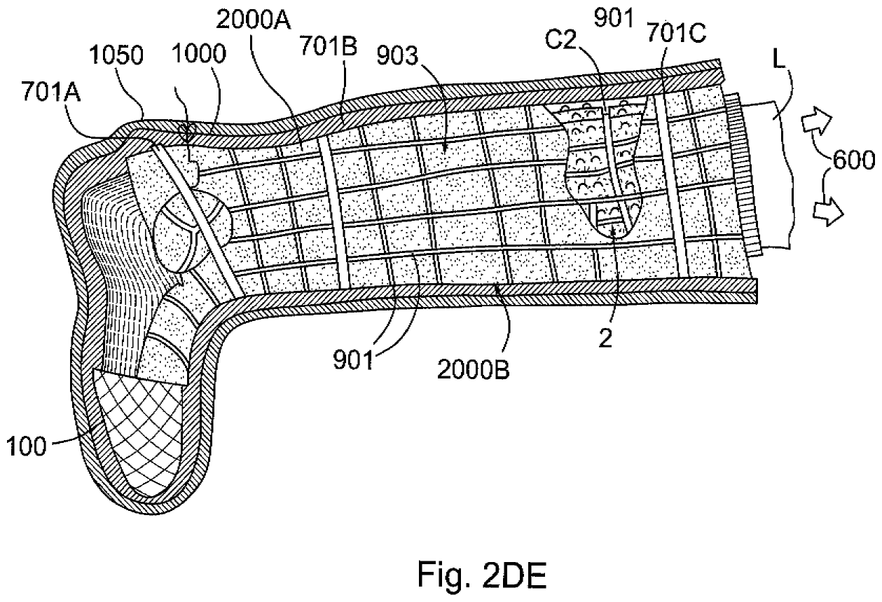

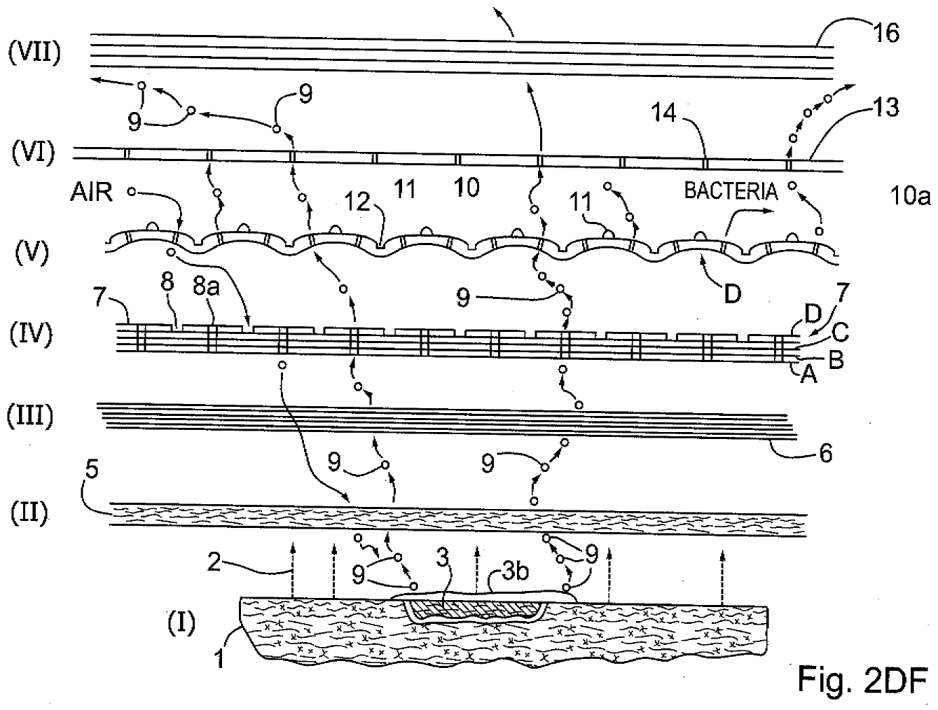

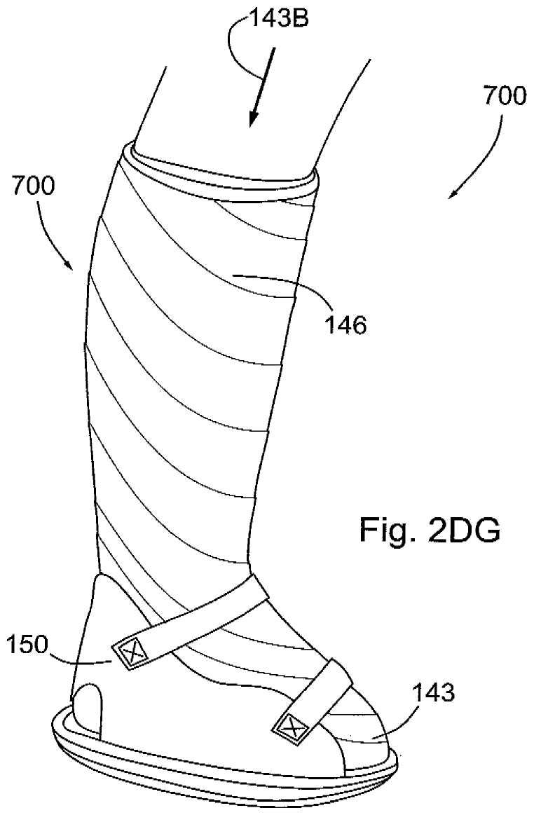

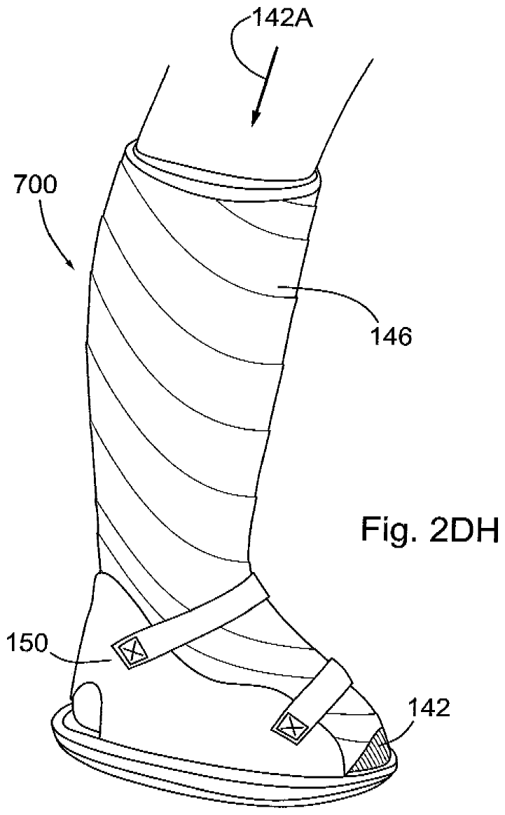

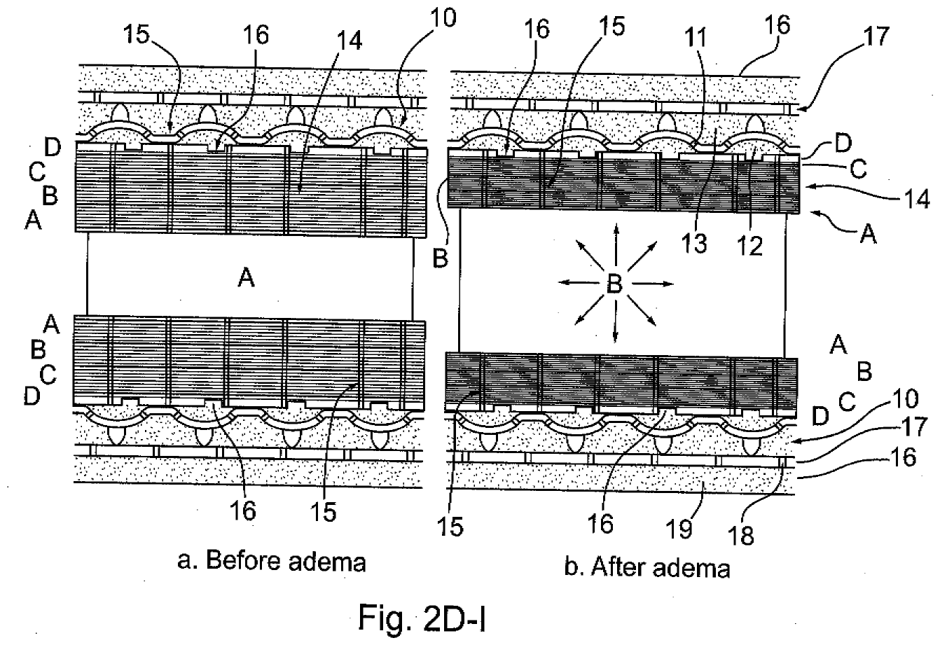

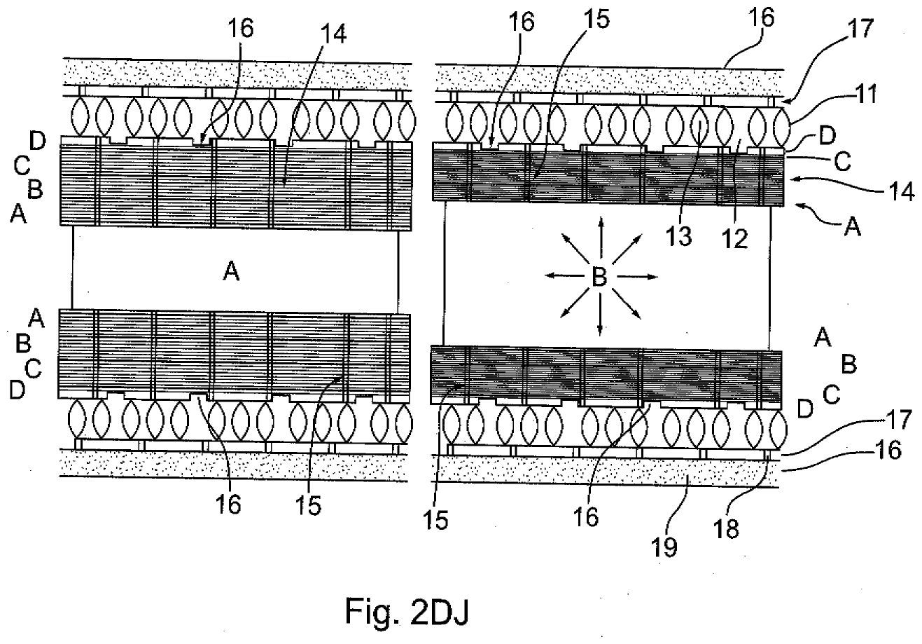

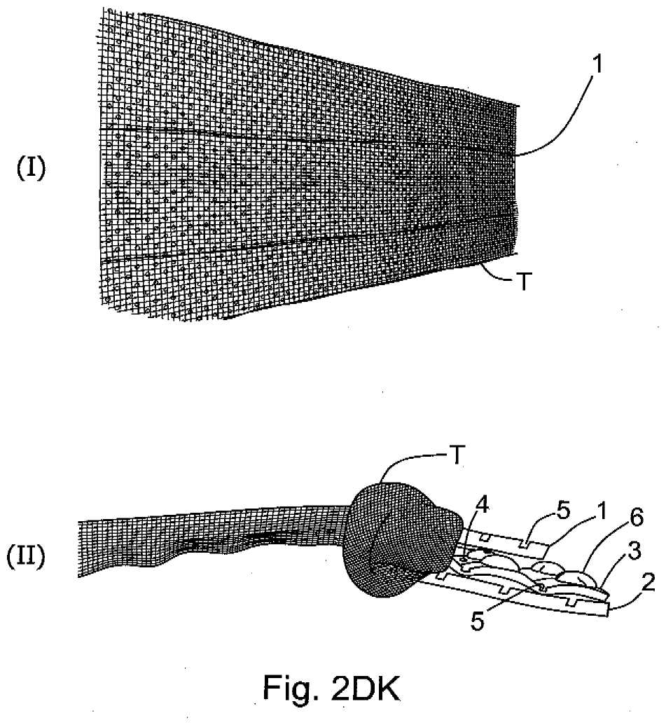

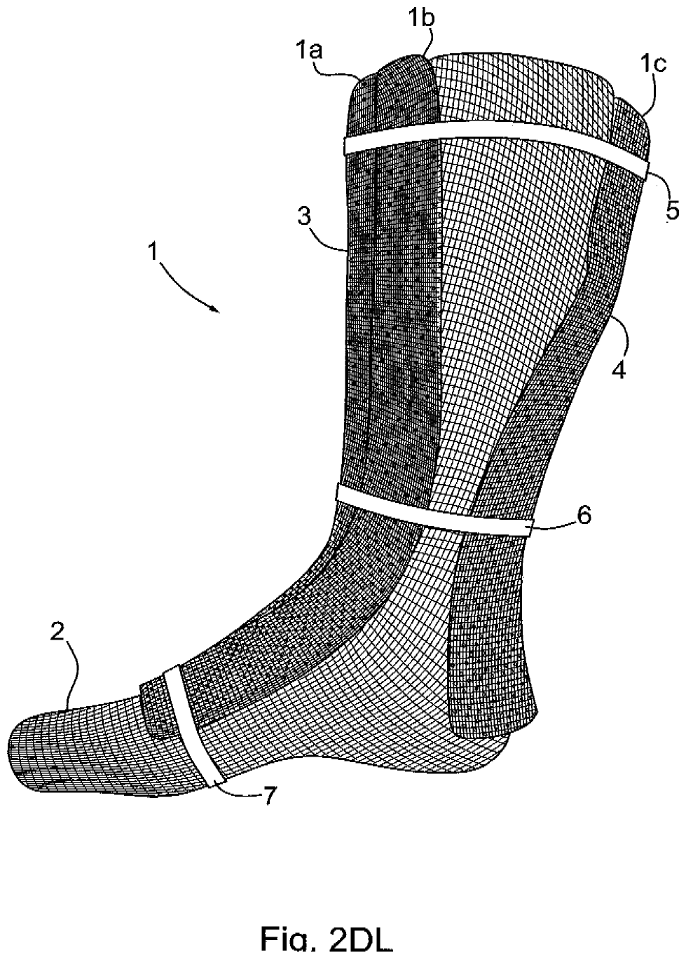

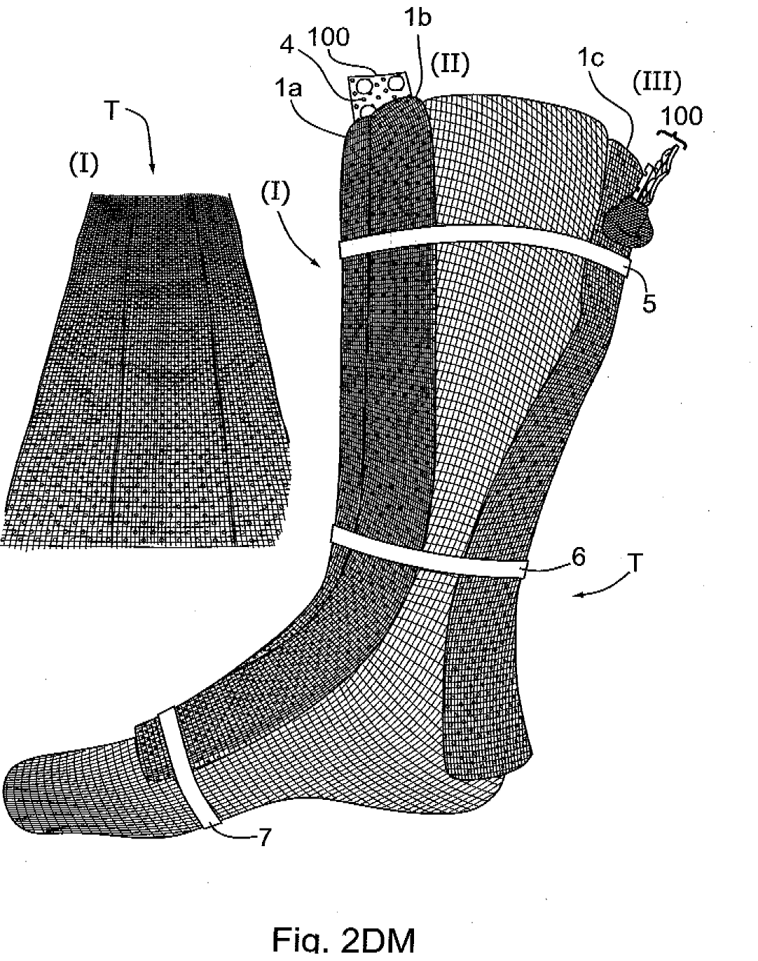

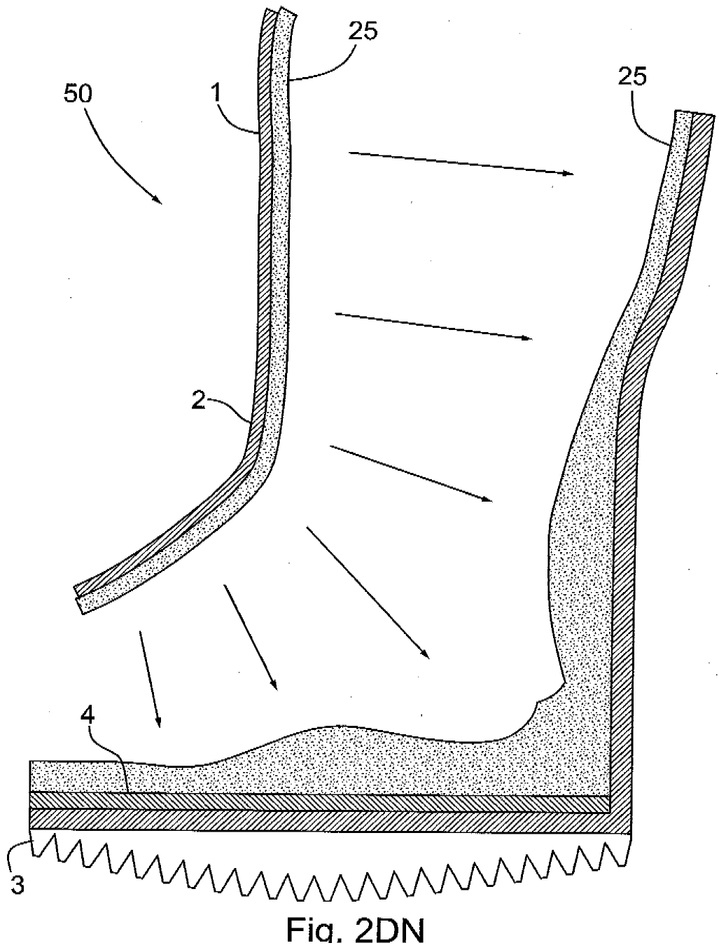

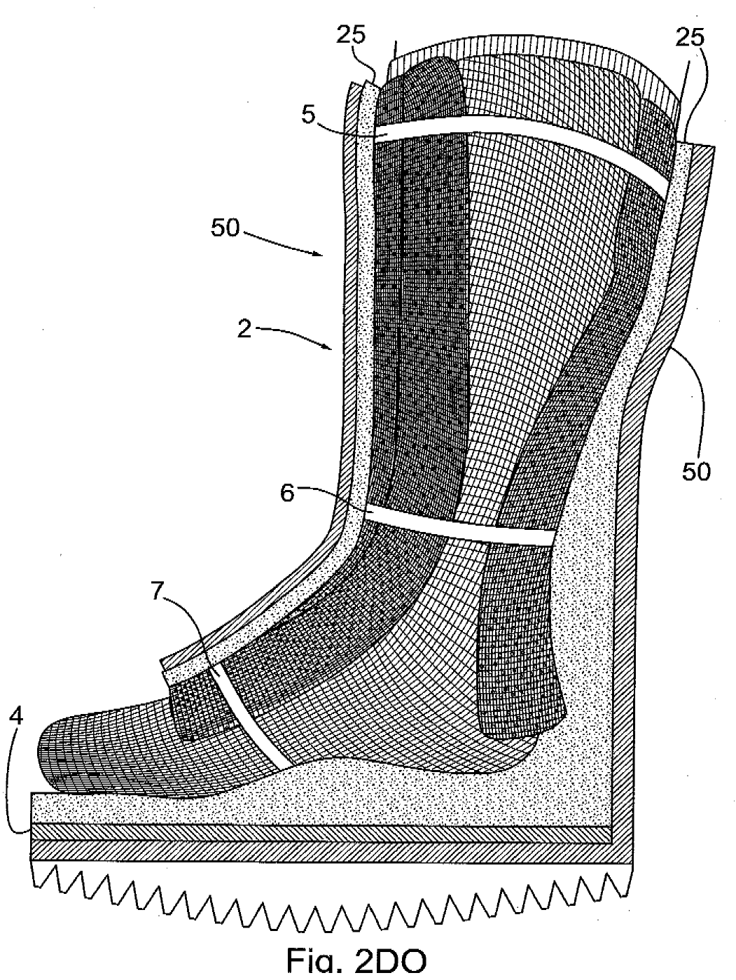

1. A method for installing a total contact cast lower leg to a patient, comprising the steps of:
(a) applying a wound care dressing to a diabetic foot ulcer patient,
(b) positioning a stockinette or sock liner over a foot and lower leg of the patient,
(c) applying a protective cover over the phalanges,
(d) selecting the adequate sizes of the venting covers to be applied on the shin and on the calf,
(e) positioning a proximal end of a compressible cover over an anterior crest of a tibia over the stockinette on the lower leg,
(f) positioning a proximal end of a compressible cover over an anterior crest of a calf over the stockinette in the lower leg,
(g) positioning a proximal end of a protruded cover over an anterior crest of a tibia over a compressible cover on the lower leg,
(h) positioning a proximal end of a protruded cover over an anterior crest of a calf over a compressible cover in the lower leg,
(i) positioning an external porous cover over a protruded cover over the tibia,
(j) positioning an external porous cover over a protruded cover over the calf,
(k) applying three layers of a fiberglass casting tape or plaster of Paris over the foot and lower leg,
(l) positioning the foot inside a cast shoe; and
(m) positioning the foot inside a removable walking brace.

2. The method of claim 1 wherein the compressible cover is made of four laminated layers of EVA™ material.

3. The method of claim 1 wherein the compressible cover is made of four laminated layers of Plastazote™ material.

4. (canceled)

5. The method of claim 1 wherein the compressible cover has one first layer closer to the wound with durometer of Shore A as 20 to 25.

6. The method of claim 1 wherein the compressible cover has a second layer on top of the first layer with durometer of Shore A as 25 to 30.

7. The method of claim 1 wherein the compressible cover has a third layer on top of the second layer with durometer of Shore A as 30 to 35.

8. The method of claim 1 wherein the compressible cover has a fourth layer on top of the third layer with durometer of Shore A as 35 to 40.

9. The method of claim 1 wherein the compressible cover the ability to store the compressive force generated by the edema of the wounded limb.

10-15. (canceled)

16. The method of claim 1 wherein the compressible cover allows excessive heat, bacteria, unwanted odors to escape the wounded zone.

17-18. (canceled)

19. The method of claim 1 wherein the compressible cover absorbs the edema compressive force sparing the need to recast the wounded leg.

20. (canceled)

21. The method of claim 1 wherein the compressible cover is a fabric spacer able to absorb the compressive force of the edema.

22. The method of claim 1 wherein the compressible cover is an antimicrobial fabric spacer of a height of 5 mm. to 7 mm.

23. The method of claim 1 wherein the compressible cover its thickness reduces as the edema force increases.

24-27. (canceled)

28. The method of claim 1 wherein the protruded layer is multi mini perforated.

29-35. (canceled)

36. The method of claim 1 wherein the protruded layer has domes for circulation of gazes and bacteria.

37. (canceled)

38. The method of claim 1 wherein the flexible layer shields the protruded layer from the wet plaster of Paris.

39-43. (canceled)

44. The method of claim 1 wherein a tubinette contains a venting system.

45-47. (canceled)

48. The method of claim 1 wherein a venting system relieves the edema pressure.

49-52. (canceled)

53. The method of claim 1 wherein a venting system alleviates mechanical load on ulcer and actively address the associated chronic oedema which can be detrimental to ulcer healing that can lead to leg's amputation.

54-58. (canceled)

59. The method of claim 1 wherein a venting system detaches separately.

60. The method of claim 1 wherein a venting system applies to a rocker walking cast shoe.

61-62. (canceled)
